(19) United States
(12) Patent Application Publication
(10) Pub. No.: US 2013/0303632 A1
(43) Pub. Date: Nov. 14, 2013

(54) CONTROLLED RELEASE SOLID DOSE FORM

(71) Applicants: Christopher J. Sewall, Hope, ME (US); William R. Blakemore, Topsham, ME (US); Bruno Leclercq, Brussels (BE)

(72) Inventors: Christopher J. Sewall, Hope, ME (US); William R. Blakemore, Topsham, ME (US); Bruno Leclercq, Brussels (BE)

(73) Assignee: FMC CORPORATION, Philadelphia, PA (US)

(21) Appl. No.: 13/890,365

(22) Filed: May 9, 2013

Related U.S. Application Data
(60) Provisional application No. 61/645,409, filed on May 10, 2012.

(51) Int. Cl. A61K 9/20

(52) U.S. Cl. CPC A61K 9/205
USPC 514/777

(57) ABSTRACT

The present invention is directed to a controlled release solid dosage form comprising a controlled release modifier and an active ingredient comprising at least one of a pharmaceutical, veterinary, or nutraceutical active ingredient, wherein the controlled release modifier comprises lambda carrageenan from the taxonomic order Halymeniales. The present invention is also directed to ethanol resistant controlled release solid dosage forms and methods for reducing the ethanol sensitivity of an active ingredient in a controlled release solid dose form.

### FIELD OF THE INVENTION

The present invention is directed to a controlled release solid dosage form comprising a controlled release modifier and an active ingredient comprising at least one of a pharmaceutical, veterinary, or nutraceutical active ingredient. The present invention is also directed to ethanol resistant controlled release solid dosage forms and methods for reducing the ethanol sensitivity of an active ingredient in a controlled release solid dose form.

### BACKGROUND OF THE INVENTION

Controlled release (referred to also as modified release) solid dosage forms generally encompass any release that is not immediate release and generally includes both extended release and enteric release. As discussed in more detail below, the controlled release solid dose forms of the present invention are extended release solid dose forms. It is an objective for extended release dose forms to achieve certain highly desired release profiles. The present invention has been found to achieve a desirable extended release profile.

In addition, an essential characteristic of all controlled release solid dosage forms is the stability and consistency of the release profile, which must be documented in regulatory applications. The design of controlled release dosage forms must mitigate the risk of premature release (“dose dumping”) leading to overdose. Solid dosage forms having unacceptable ethanol sensitivity have been withdrawn from the market. The inventors have surprisingly found that the addition of the specific controlled release modifier of the invention provides a relatively high degree of ethanol resistance.

### SUMMARY OF THE INVENTION

The present invention is directed to a controlled release solid dose form comprising a controlled release modifier and an active ingredient, wherein the controlled release modifier comprises lambda carrageenan from the taxonomic order Halymeniales and the active ingredient comprises at least one of a pharmaceutical, veterinary, or nutraceutical active ingredient.

The present invention is also directed to ethanol resistant controlled release solid dosage forms comprising the controlled release modifier of the invention, as well as methods for reducing the ethanol sensitivity of an active ingredient in a controlled release solid dose form.

### BRIEF DESCRIPTION OF THE FIGURES

FIG. 1 is a graph showing the dissolution testing of controlled release theophylline tablets (compressed at 12 kN). See Example 1.

FIG. 2 is a graph showing the dissolution testing of controlled release theophylline tablets (compressed at 8 kN). See Example 2.

### DETAILED DESCRIPTION OF THE INVENTION

The present invention is directed to a controlled release solid dose form comprising a controlled release modifier and an active ingredient, wherein the controlled release modifier comprises lambda carrageenan from the taxonomic order Halymeniales and the active ingredient comprises at least one of a pharmaceutical, veterinary, or nutraceutical active ingredient.

As used herein, “controlled release” and “modified release” mean a release of an active ingredient that is extended (also referred to as “sustained”) release and not immediate release or enteric release. That is, immediate release of a drug is often considered to be greater than 80% of the drug released in less than 15 minutes when measured in vitro in accordance with the following standard test: the dosage form is exposed to 900 mL 0.1 N HCl in a USP 32 paddle apparatus (37° C., 100 rpm). At pre-determined time points, samples are withdrawn and their drug contents analyzed using an appropriate analytical technique for the respective drug. Controlled release and modified release, as used herein, mean extended release profiles that (when measured in the foregoing test) are i) not immediate release and include less than 80% drug released in greater than 15 minutes, and ii) >10% release of the active at two hours.

The controlled release solid dose form can be any conventional solid dose form that is orally administered including tablets, pellets or granules. The solid dose form may be made by any conventional technique for making such dose forms, such as direct compression, dry granulation and wet granulation.

The controlled release solid dose form can contain any components that are commonly used in such dose forms, such as excipients. Examples of conventional excipients include binders (such as microcrystalline cellulose), disintegrants, diluents, lubricants, glidants, matrix formers, emulsifying-solubilizing agents, sweetening agents, coating agents, antimicrobial preservatives, etc.

The controlled release modifier comprising lambda carrageenan from the taxonomic order Halymeniales is present in an amount that is effective to control the release of the active ingredient. This amount can vary depending on the components used in the dose form and the specific controlled release rate that is targeted. In general, such an amount can be at least 10% by weight of the solid dosage form, more particularly, at least 15%, or at least 18% by weight of the solid dosage form. Such an amount may also be 10-60% by weight of the solid dosage form, more particularly, 15-40%, 15-30%, or 18-22% by weight of the solid dosage form.

The controlled release modifier of the present invention comprises lambda carrageenan from at least one seaweed of the taxonomic order Halymeniales. The lambda carrageenan may be from at least one algal family comprising Halymeniaceae or Tsenglaceae. The seaweed may be from at least one genus comprising Halymenia, Grateloupia, or Tsenglia. The seaweed may be from at least one algal species comprising Halymenia durvillei, Halymenia floresii, Halymenia fimbriata, or Grateloupia filicina. Additional basionym species of Halymenia durvillei include Halymenia formosa, Halymenia fimbriata, and Halymenia microcarpa; additional basionym species of Halymenia floresii include Halymenia venusta; and additional basionym species of Grateloupia filicina include Grateloupia subpectinata, Grateloupia conferta, Grateloupia porracea, Grateloupia filiformis, Grateloupia catenata, and Grateloupia prolongata.

The lambda carrageenan from Halymeniales has a different chemical structure than other traditional commercial sources of lambda carrageenans. For example, lambda carrageenan from traditional commercial sources such as Chondrus crispus, Gigartina skottsbergii, and Sarcothalia crispata have exclusively a D-galactan molecular backbone structure (with no L-galactose present). Compared to such traditional lambda carrageenans, the lambda carrageenan from Halymeniales has less ester sulfate content, more pyruvate content, increased methyl content and the presence of L-galactose (with D-galactose forming a DL-hybrid galactan molecular backbone). In regard to the lambda carrageenan from Halymeniales, the pyruvate ester, methyl ester, and L-galactose distributions appear to be random along the molecular backbone, but each are associated with specific carrageenan moieties. The pyruvate is present as galactose-2-sulphate-4:6-pyruvate, the methyl as galactose-6-methyl, and the L-galactose as unsulphated 3:6-anhydrogalactose. In addition, the ester sulphate content of carrageenans ranges from 18-40%. The lambda carrageenan from Halymenia durvillei has been found to have an ester sulphate content in the range 26-34% as compared to regular lambda carrageenans having an ester sulphate content in the range of 34-40%. The lambda carrageenan from Sarcothalia crispata is the closest in nature to the ideal structure for lambda carrageenan as detailed in the literature, and, hence, it is commonly used as a reference material when assessing the functionality of various lambda carrageenans. The ester sulphate content in the lambda carrageenan from Halymenia durvillei is lower at 32-36% when compared to the content in Sarcothalia crispata of 38%.

The lambda carrageenan from Halymeniales comprises at least 75%, at least 90%, at least 95% and at least 100% of all lambda carrageenan used in the present invention as a controlled release (i.e., extended release) modifier.

The solid dose forms of the present invention are found to have high levels of ethanol resistance. This means that the release kinetics of the active ingredient are not significantly affected by the presence of alcohol in accordance with the following definition. More specifically, as used herein, a solid dosage form is highly ethanol resistant (or not sensitive to ethanol) if the in vitro drug release data in 0.1 N HCl is compared with and without 40% ethanol for 2 hours at 37° C. and the difference throughout the two hour period in release profiles between the ethanol free media and ethanol containing media is less than 10%, more preferably, less than 5%, when less than 50%, more particularly, from 1 to 40%, of the active is released in the ethanol free media in two hours. A typical test and apparatus for determining the foregoing dissolution profile is USP 4, 12 mm cell, 8 mL, 900 ml 0.1 N HCl, 37° C. with and without 40% ethanol for two hours.

As a preferred embodiment, in addition to being ethanol resistant throughout the two hour period in 0.1 N HCl as noted in the foregoing definition, the present invention has also been found to be highly ethanol resistant when subsequently and immediately placed (after the two hour period in 0.1 N HCl) in 0.05M phosphate buffer at pH 6.8 at 37° C., e.g., for at least four hours, at least five hours, or at least six hours. More specifically, the difference in release profiles (between the sample exposed to ethanol containing media and the sample exposed to ethanol free media in the foregoing 0.01 N HCl two hour test) when subsequently placed in 0.05 M phosphate buffer at pH 6.8 at 37° C. is less than 15% for at least four hours, at least five hours, or at least six hours. A typical test and apparatus for determining the dissolution in phosphate buffer is USP 4, 12 mm cell, 8 mL/min, 900 mL of 0.05M phosphate buffer, pH 6.8, 37° C.

The foregoing levels of ethanol resistance that are achieved by the present invention are very high levels of ethanol resistance and are significantly higher than many standards for ethanol resistance in general.

The present invention is also directed to a method of reducing the ethanol sensitivity of a solid dosage form comprising adding the controlled release modifier of the invention to a solid dosage form comprising a pharmaceutical, nutraceutical or veterinary active ingredient.

A wide variety of therapeutically active agents can be used in conjunction with the present invention. The therapeutically active agents (e.g. pharmaceutical agents) which may be used in the compositions of the present invention include both water soluble and water insoluble drugs. Examples of such therapeutically active agents include antihistamines (e.g., dimenhydrinate, diphenhydramine, chlorpheniramine and dexchlorpheniramine maleate), analgesics (e.g., aspirin, codeine, morphine, dihydromorphone, oxycodone, etc.), anti-inflammatory agents (e.g., naproxyn, diclofenac, indomethacin, ibuprofen, acetaminophen, aspirin, sulindac), gastro-intestinals and anti-emetics (e.g., metoclopramide), anti-epileptics (e.g., phenytoin, meprobamate and nitrezepam), vasodilators (e.g., nifedipine, papaverine, diltiazem and nicardirine), anti-tussive agents and expectorants (e.g., codeine phosphate), anti-asthmatics (e.g. theophylline), anti-spasmodics (e.g. atropine, scopolamine), hormones (e.g., insulin, leparin), diuretics (e.g., eltacrymic acid, bendrofluazide), anti-hypotensives (e.g., propranolol, clonidine), bronchodilators (e.g., albuterol), anti-inflammatory steroids (e.g., hydrocortisone, triamcinolone, prednisone), antibiotics (e.g., tetracycline), antihemorrhoidals, hypnotics, psychotropics, antidiarrheals, mucolytics, sedatives, decongestants, laxatives, antacids, vitamins, stimulants (including appetite suppressants such as phenylpropanolamine) and mixtures thereof. The above list is not meant to be exclusive.

In general, the amount of the active ingredient useful in the present invention is from 2-80% by weight of the dose form, more particularly, from 20-70% by weight of the dose form, more particularly, from 30-70% by weight of the dose form.

The present invention is now described in more detail by reference to the following examples, but it should be understood that the invention is not construed as being limited thereto. Unless otherwise indicated herein, all parts, percents, ratios and the like are by weight.

### EXAMPLES

Tablets were prepared in Examples 1, 2 and 3 using the following process. All formulation ingredients (except for the magnesium stearate) described below were weighed and premixed in a V-blender for 15 minutes. The magnesium stearate was then added followed by two additional minutes of mixing. The mixture was compressed on a Stokes 512 Tablet Press with four stations. Standard 7/16″ concave punches and corresponding dies were used. Tablet weight was adjusted to 500 mg. SMI Director™ data acquisition system was used to record the compaction process. Viscosities of the carrageenan samples were measured as follows: a 1.5% by weight aqueous solution of the carrageenan sample was prepared by mixing and heating to 85° C., holding for 15 minutes, and then cooling to 75° C. before measuring the viscosity using a Brookfield LV viscometer.

### Example 1

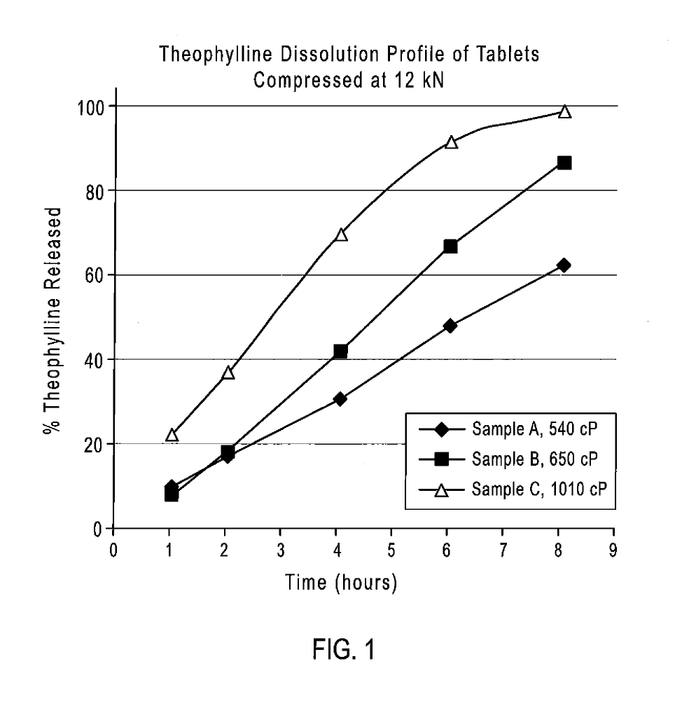

Dissolution testing of controlled-release theophylline tablets (500-mg claim weight) was performed to evaluate the use of lambda carrageenan of the invention as a controlled release modifier. Tablets were prepared using Samples A (commercially available lambda carrageenan), B (commercially available lambda carrageenan) and C (inventive carrageenan) below using the direct compression method described above. The dissolution testing used a USP apparatus 1 (basket), 100 rpm, 900 mL, 0.05M phosphate buffer, 37° C., pH 7.5; UV, 271 nm. The results are set forth in Table 1 and FIG. 1.
Sample A: Theophylline extended-release tablets (500-mg claim weight) were prepared as noted above containing 60.0% anhydrous theophylline (Spectrum, ZH0127), 19.0% Avicel® PH-102 MCC (FMC), 20.0% commercially available lambda carrageenan (FMC; viscosity: 540 cP, pH 9.4) and 1.0% magnesium stearate (Mallinckrodt). Tablets were produced using a 12 kN compression force.
Sample B: Tablets were prepared in the same manner as Sample A, but the commercially available lambda carrageenan was replaced with 20.0% of a lambda carrageenan (i.e., about 95-98% lambda carrageenan and about 2-5% kappa-2 carrageenan) extracted from Sarcothalia crispata (viscosity: 650 cP, pH 9.7).
Sample C: Tablets were prepared in the same manner as Sample A, but the commercially available lambda carrageenan was replaced with 20.0% of the lambda carrageenan of the invention (viscosity: 1010 cP, pH 9.7).

TABLE 1
Dissolution Testing of Extended-Release Theophylline Tablets

| | Mean Percent Release ± S.D. (n = 6) | | |
|---|---|---|---|
| Time | Sample A | Sample B | Sample C |
| (Hours) | (comparative) | (comparative) | (invention) |
| 1 | 10 ± 0.4 | 8 | 22 ± 0.5 |
| 2 | 17 | 18 ± 0.9 | 37 ± 1.0 |
| 4 | 31 ± 1.0 | 42 ± 2.2 | 70 ± 1.9 |
| 6 | 48 ± 5.6 | 67 ± 3.0 | 92 ± 1.2 |
| 8 | 62 ± 5.8 | 87 ± 2.7 | 99 ± 0.8 |
| Infinity | 74 ± 6.3 | 99 ± 1.0 | 100 ± 0.8 |
| (200 rpm/2 Hrs) | | | |

FIG. 1 is a graph containing the data in Table 1. Tablets made from all three samples showed controlled release behavior, but there are at least two important and significant differences. First, the speed of release was slowest to fastest from (in order) tablets containing Samples A, B and C. Second, as can be seen in Table 1 and FIG. 1, tablets made from Sample C showed 100% dissolution at 8 hours as opposed to the significantly less dissolution at 8 hours for Samples A and B.

### Example 2

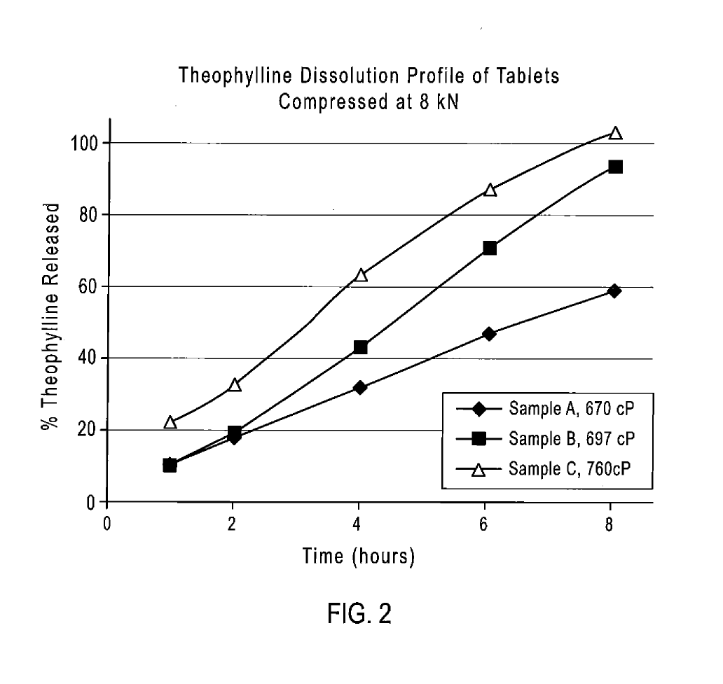

Dissolution testing of modified-release theophylline tablets (500-mg claim weight) was performed to evaluate the use of lambda carrageenan of the invention as a controlled release modifier. Tablets were prepared using Samples A (commercially available lambda carrageenan), B (commercially available lambda carrageenan) and C (inventive carrageenan) below using the direct compression method described above. The dissolution testing used a USP apparatus 1 (basket), 100 rpm, 900 mL, 0.05M phosphate buffer, 37° C., pH 7.5; UV, 271 nm. The results are set forth in Table 2 and FIG. 2.
Sample A: Theophylline modified-release tablets (500-mg claim weight) were prepared containing 60.0% anhydrous theophylline (Spectrum, ZH0127), 19.0% Avicel® PH-102 MCC (FMC), 20.0% commercially available lambda carrageenan (FMC; viscosity 670 cP, pH 9.2) and 1.0% magnesium stearate (Mallinckrodt-Baker). Tablets were produced with a 7.9 kN compaction force.
Sample B: Tablets were prepared in the same manner as Sample A, but the commercially available lambda carrageenan was replaced with 20.0% of a lambda carrageenan (i.e., about 95-98% lambda carrageenan and about 2-5% kappa-2 carrageenan) extracted from Sarcothalia crispata (viscosity 697 cP, pH 9.4). Tablets were produced with an 8.0 kN compaction force.
Sample C: Tablets were prepared in the same manner as Sample A, but the commercially available lambda carrageenan was replaced with 20.0% of lambda carrageenan of the invention (viscosity 760 cP, pH 9.7). Tablets were produced with an 8.0 kN compaction force.

TABLE 2
Dissolution Testing of Modified-Release Theophylline Tablets

| | Mean Percent Release ± S.D. (n = 3) | | |
|---|---|---|---|
| Time | Sample A | Sample B | Sample C |
| (Hours) | (comparative) | (comparative) | (invention) |
| 1 | 11 | 10 | 22 ± 1.0 |
| 2 | 18 ± 0.6 | 19 ± 0.6 | 33 ± 1.0 |
| 4 | 32 ± 2.0 | 43 ± 1.0 | 63 ± 2.6 |
| 6 | 47 ± 2.5 | 71 ± 1.0 | 87 ± 1.0 |
| 8 | 59 ± 3.5 | 94 ± 2.3 | 103 ± 0.6 |
| Infinity | 71 ± 7.6 | 103 ± 1.0 | |
| (200 rpm/2 Hrs) | | | |

FIG. 2 is a graph containing the data in Table 2. As in Example 1, the tablets made from all three samples showed controlled release behavior, but there are at least two important and significant differences. First, the speed of release was slowest to fastest from (in order) tablets containing Samples A, B and C. Second, as can be seen in Table 2 and FIG. 2, tablets made from Sample C showed about 100% dissolution at 8 hours as opposed to the significantly less dissolution at 8 hours for Samples A and B.

### Example 3

### Ethanol Resistance Study

Dissolution testing of controlled-release theophylline tablets (300-mg claim weight) was performed to evaluate the ethanol resistance of solid dosage forms containing the lambda carrageenan of the invention and compared to a control in a model study. Tablets were prepared using Samples A (commercially available lambda carrageenan), B (inventive carrageenan) and C (inventive carrageenan) below using the direct compression method described above. The dissolution testing used USP apparatus 4, 12 mm cell, 8 mL/min, 900 mL 0.1N HCl 37° C. with and without 40% ethanol for the first two hours and the media was then replaced with 900 mL of 0.05M phosphate buffer, pH 6.8 at 37° C. for the remainder of the tested time period. The results are set forth in Tables 3, 4 and 5.
Sample A: Control. Theophylline modified-release tablets (300-mg claim weight) containing 60.0% theophylline (Spectrum), 19.0% Avicel® PH-102 MCC (FMC Corporation), 20.0% commercially available lambda carrageenan (FMC Corporation) and 1.0% magnesium stearate (Mallinckrodt-Baker). Tablets produced using a 12 kN compaction force.
Sample B: Same as Sample A, but with 20.0% lambda carrageenan of the invention replacing the commercially available lambda carrageenan of the control.
Sample C: Same as Sample A, but with 20.0% lambda carrageenan of the invention replacing the commercially available lambda carrageenan of the control.

TABLE 3
Sample A
Dissolution Testing of Modified-Release Theophylline Tablets

Mean Percent
Release ± S.D. (n = 3)

| Time | 0.1N HCl/ | 0.1N HCl with |
|---|---|---|
| (Hours) | 2 Hrs | 40% Ethanol/2 Hrs |
| 0.5 | 12 | 9 ± 1.2 |
| 1 | 21 ± 0.6 | 15 ± 2.1 |
| 1.5 | 31 ± 1.5 | 22 ± 2.1 |
| 2 | 42 ± 3.8 | 30 ± 2.3 |
| 4 | 61 ± 6.4 | 44 ± 2.6 |
| 6 | 71 ± 6.9 | 54 ± 2.1 |
| 8 | 79 ± 8.1 | 62 ± 3.5 |
| 10 | 84 ± 8.7 | 66 ± 3.6 |
| 12 | 88 ± 8.4 | 73 ± 8.7 |

TABLE 4
Sample B
Dissolution Testing of Modified-Release Theophylline Tablets

Mean Percent
Release ± S.D. (n = 3)

| Time | 0.1N HCl/ | 0.1N HCl with |
|---|---|---|
| (Hours) | 2 Hrs | 40% Ethanol/2 Hrs |
| 0.5 | 11 ± 0.6 | 12 ± 0.6 |
| 1 | 18 ± 0.6 | 19 ± 0.6 |
| 1.5 | 24 ± 0.6 | 25 ± 1.0 |
| 2 | 30 ± 0.6 | 30 ± 0.6 |
| 4 | 41 ± 2.1 | 44 ± 0.6 |
| 6 | 49 ± 4.0 | 54 ± 2.1 |
| 8 | 56 ± 6.0 | 62 ± 3.1 |
| 10 | 62 ± 7.0 | 66 ± 4.2 |
| 12 | 67 ± 8.5 | 72 ± 4.6 |

TABLE 5
Sample C
Dissolution Testing of Modified-Release Theophylline Tablets

Mean Percent
Release ± S.D. (n = 3)

| Time | 0.1N HCl/ | 0.1N HCl with |
|---|---|---|
| (Hours) | 2 Hrs | 40% Ethanol/2 Hrs |
| 0.5 | 12 ± 2.1 | 14 ± 1.0 |
| 1 | 21 ± 2.5 | 22 ± 1.5 |
| 1.5 | 27 ± 3.0 | 28 ± 1.7 |
| 2 | 33 ± 3.5 | 35 ± 2.3 |
| 4 | 47 ± 3.6 | 51 ± 3.6 |
| 6 | 60 ± 4.2 | 61 ± 4.0 |
| 8 | 74 ± 4.0 | 71 ± 6.4 |
| 10 | 89 ± 6.6 | 82 ± 8.6 |
| 12 | 101 ± 7.5 | 94 ± 11.5 |

Sample A displayed an 80% controlled release of the drug at eight hours in dissolution media of 0.1N HCl for the first two hrs followed by six hrs in 0.05M phosphate buffer with pH 6.8. Sample A showed slower drug dissolution (62% in eight hrs) in the presence of 40% ethanol (i.e., first two hours in 0.1N HCl with 40% ethanol followed by four hours in 0.05 M phosphate buffer at pH 6.8). Samples B and C displayed 56% and 74% controlled release of the drug at eight hours, respectively (when tested in 0.1N HCl for the first two hours followed by six hours in 0.05 M phosphate buffer at pH 6 8) Importantly, Samples B and C unexpectedly showed a very high level of ethanol resistance when compared to Sample A; e.g., Samples B and C were so highly ethanol resistant that their dissolution profiles in ethanol were almost the same as the dissolution profile in no alcohol.

While the invention has been described in detail and with reference to specific embodiments thereof, it will be apparent to one skilled in the art that various changes and modifications can be made therein without departing from the spirit and scope thereof.

What is claimed is:

1. A controlled release solid dose form comprising a controlled release modifier and an active ingredient, wherein said controlled release modifier comprises lambda carrageenan from the taxonomic order Halymeniales and said active ingredient comprises at least one of a pharmaceutical, veterinary, or nutraceutical active ingredient.

2. The controlled release solid dose form of claim 1, wherein the solid dosage form is ethanol resistant.

3. The controlled release solid dose form of claim 2, wherein: i) the difference in the in vitro active release data when placed in 0.1 N HCl for 2 hours at 37° C. with and without 40% ethanol is less than 10% when less than 50% of the active is released in ethanol free media at two hours, and ii) when subsequently and immediately placed in 0.05 M phosphate buffer at pH 6.8 at 37° C. for at least four hours, the difference in release profiles between the samples placed in 0.1 N HCl in ethanol free media and ethanol containing media throughout the four hour period in said 0.05M phosphate buffer is less than 15%.

4. The controlled release solid dose form of claim 1, wherein said dose form is a tablet, pellets or granules.

5. The controlled release solid dosage form of claim 1, wherein said controlled release modifier is present in an amount of 10-60% by weight of the solid dosage form.

6. The controlled release solid dosage form of claim 1, wherein said controlled release modifier is present in an amount of 15-40% by weight of the solid dosage form.

7. The controlled release solid dosage form of claim 1, wherein said controlled release modifier is present in an amount of 15-30% by weight of the solid dosage form.

8. The controlled release solid dosage form of claim 1, wherein said controlled release modifier is present in an amount of 18-22% by weight of the solid dosage form.

9. The controlled release solid dose form of claim 1, wherein said Halymeniales comprises seaweed from the family Halymeniaceae.

10. The controlled release solid dose form of claim 9, wherein said seaweed is from a genus of least one of Halymenia or Grateloupia.

11. The controlled release solid dose form of claim 10, wherein said seaweed is from a species of at least one of Halymenia durvillei, Halymenia floresii, or Grateloupia filicina.

12. The controlled release solid dose form of claim 11, wherein said species is Halymenia durvillei.

13. The controlled release solid dose form of claim 1, wherein said active ingredient is present in an amount of from 2-80% by weight of said dose form.

14. A method of reducing the ethanol sensitivity of a pharmaceutical, nutraceutical or veterinary active ingredient in a controlled release solid dosage form comprising adding a controlled release modifier to said controlled release solid dosage form, wherein said controlled release modifier comprises lambda carrageenan from the taxonomic order Halymeniales.

15. The method of claim 14, wherein: i) the difference in the in vitro active release data when placed in 0.1 N HCl for 2 hours at 37° C. with and without 40% ethanol is less than 10% when less than 50% of the active is released in ethanol free media at two hours, and ii) when subsequently and immediately placed in 0.05 M phosphate buffer at pH 6.8 at 37° C. for at least four hours, the difference in release profiles between the samples placed in 0.1 N HCl in ethanol free media and ethanol containing media throughout the four hour period in said 0.05M phosphate buffer is less than 15%.
